(19) United States
(12) Patent Application Publication
(10) Pub. No.: US 2015/0266945 A1
(43) Pub. Date: Sep. 24, 2015

(54) FUSION PROTEIN, A METHOD OF MAKING THE SAME, AND A METHOD OF DELIVERING ANTIGENIC PEPTIDE INTO THE ENDOPLASMIC RETICULUM BY THE FUSION PROTEIN

(71) Applicant: Dalin Tzu Chi Hospital, Buddhist Tzu Chi Medical Foundation, Dalin Township (TW)

(72) Inventors: Ning-Sheng LAI, Chiayi County (TW); Hsien-Bin HUANG, Chiayi County (TW); Hui-Chun YU, Chiayi County (TW)

(73) Assignee: Dalin Tzu Chi Hospital, Buddhist Tzu Chi Medical Foundation, Dalin Township (TW)

(21) Appl. No.: 14/222,228

(22) Filed: Mar. 21, 2014

(51) Int. Cl. C07K 16/18

(52) U.S. Cl. CPC C07K 16/18; C07K 2319/10

(57) ABSTRACT

A method of delivering antigenic peptide into the endoplasmic reticulum by the fusion protein includes: (a) preparing a fusion protein with a Tat-derived peptide, a (His)6 peptide, a ubiquitin, and an antigenic peptide provided in turn from the N-terminal end to the C-terminal end; (b) delivering the fusion protein across the cell membrane into the cytosol through the Tat-derived peptide; (c) cleaving the antigenic peptide from the fusion protein by cytosolic ubiquitin C-terminal hydrolases; and (d) transporting the antigenic peptide into the endoplasmic reticulum (ER) by a TAP1/2 transporter on the ER membrane.

### BACKGROUND OF THE INVENTION

1. Field of the Invention

The present invention relates to a method of delivering antigenic peptide into the endoplasmic reticulum (ER) by the fusion protein. The method is allowed to be used in clinical research of treatment for Ankylosing Spondylitis.

2. Description of the Related Art

Pathogeny mechanism of Ankylosing Spondylitis (AS) relates to the structural variation of human leukocytic antigen-B27 (HLA-B27), wherein HLA-B27 is a molecular of major histocompatibility complex class I (MHC class I), which consists of a section of heavy chain (HC) and a β₂-microglobin (β₂m). When being synthesized in the endoplasmic reticulum (ER), HLA-B27 is complexed with antigenic peptide into a complex. Afterward, the complex is transported to the cellular membrane by the trans-Golgi, and the antigenic peptide is presented on the cellular surface.

Being indicated by researches conducted by Kollnberger S, et al (Arthritis Rheum, 46:2972-82, 2002) and Dangoria N S, et al (J Biol Chem, 277:23459-68, 2002), the heavy chain of HLA-B27, before being complexed with β₂m and antigenic peptide in the endoplasmic reticulum, tends to naturally fold slowly, while the misfolded heavy chain of HLA-B27 may accumulate in the endoplasmic reticulum, resulting in ER stress and forming a HLA-B27 heavy chain homodimer, (B27-HC)₂, whereby a (B27-HC) is not going to be complexed with a β₂m. When a (B27-HC)₂ is presented on the cellular membrane, natural killer-cells (NK cells) and T-helper 17 cells are activated to trigger the inflammatory response, which is the main potential pathogeny cause of Ankylosing Spondylitis (J Immunol, 173:1699-710, 2004; J Immunol, 186:2672-80, 2011).

Furthermore, it is indicated that the antigenic peptide complexed with HLA-B27 in the endoplasmic reticulum is beneficial for the heavy chains of HLA-B27 to correctly fold (J Immunol, 163:6665-70, 1999; J Exp Med, 180:2163-71, 1994), Therefore, the heavy chain of HLA-B27 becomes the target objective of important medication used for treating Ankylosing Spondylitis. For example, antigenic peptides complexed with HLA-B27 are efficiently transported to the endoplasmic reticulum for facilitating the folding of the heavy chains of HLA-B27, becoming a potential mechanism for Ankylosing Spondylitis treatment. However, in relative researches of the present invention, no appropriate techniques for transporting antigenic peptides with accurate size and sequence into the endoplasmic reticulum are developed.

### SUMMARY OF THE INVENTION

For improving such difficulties, the present invention provides a method of delivering antigenic peptide with accurate size and sequence into the endoplasmic reticulum by the fusion protein, whereby facilitating the heavy chains of HLA-B27 correctly folded in the endoplasmic reticulum.

For achieving the aforementioned objective, the present invention provides a method of delivering antigenic peptide into the endoplasmic reticulum by the fusion protein, comprising following steps:

(a) Preparing a fusion protein, wherein the sequence of the fusion protein is provided in turn with a Tat-derived peptide, a (His)₆ peptide, a ubiquitin, and an antigenic peptide from the N-terminal end to the C-terminal end;

(b) delivering the fusion protein across the cell membrane into the cytosol by the Tat-derived peptide;

(c) cleaving the antigenic peptide from the fusion protein by cytosolic ubiquitin C-terminal hydrolases; and

(d) transporting the cleaved antigenic peptide into the endoplasmic reticulum by a TAP1/2 transporter on the endoplasmic reticulum membrane.

The present invention provides a method of effectively delivering antigenic peptide into the endoplasmic reticulum. The antigenic peptide thus delivered facilitates the correct folding process of the heavy chains of HLA-B27, which helps decrease the amounts of (B27-HC)₂, thereby mitigating the inflammatory response. Therefore, the present invention is allowed to be applied to clinical researches of treating Ankylosing Spondylitis (AS).

Furthermore, the present invention is applicable to cancer treatment researches. If antigenic peptide derived from bacteria is delivered into the endoplasmic reticulum of human cells, the antigenic peptide is presented on the cellular surface by the HLA-B27 of the human cells, whereby the CD8⁺ T cell is activated and the cytotoxicity response is thereby triggered. Therefore, the present invention is applicable to cancer treatment researches. While delivering a specific antigenic peptide by such method into cancer cells, the MI-IC class I molecules of the cancer cells thus presents the antigenic peptide delivered on the cellular membrane thereof, whereby the cancer cells are toxified by the activated CD8⁺ T cells and the cancer is being treated.

### BRIEF DESCRIPTION OF THE DRAWINGS

FIG. 1 is a flow chart of delivering antigenic peptide in accordance with the present invention.

FIG. 2 is a structural diagram of the fusion protein in accordance with the present invention.

FIG. 3 is another structural diagram of the fusion protein in accordance with the present invention.

FIG. 4A to FIG. 4D are statistic diagrams illustrating the reduction of (B27-HC)₂ while the fusion protein is delivered into cells.

FIG. 5A to FIG. 5C are statistic diagrams illustrating the antigenic peptide presented on the surface of a cell.

FIG. 6A to FIG. 6C are statistic diagrams illustrating the amounts of cells with apoptosis after the fusion protein is delivered into cells.

### DETAILED DESCRIPTION OF THE INVENTION

The aforementioned and further advantages and features of the present invention will be understood by reference to the description of the preferred embodiment in conjunction with the accompanying drawings.

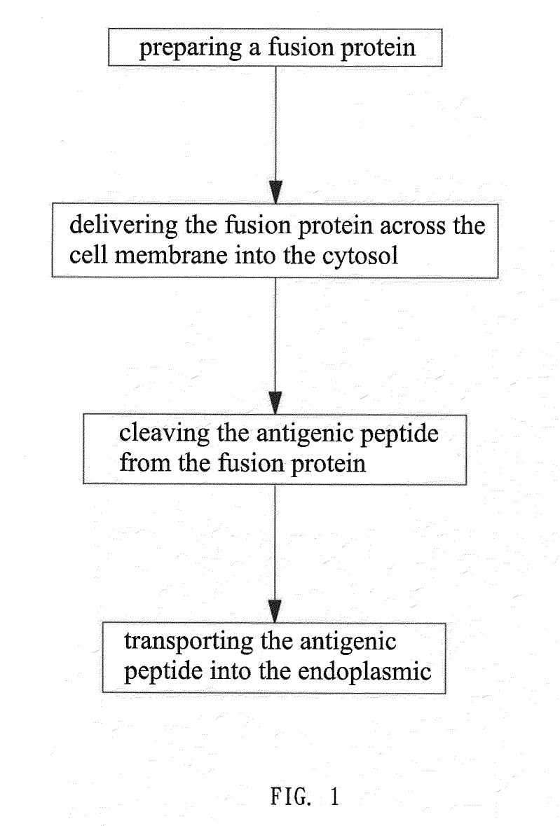

Referring to FIG. 1 to FIG. 3, the present invention provides a method of delivering antigenic peptide into the endoplasmic reticulum by the fusion protein, comprising:

(a) preparing a fusion protein 100;

(b) delivering the fusion protein 100 across the cellular membrane into the cytosol;

(c) cleaving the antigenic peptide 40; and transporting the antigenic peptide 40 into the endoplasmic reticulum.

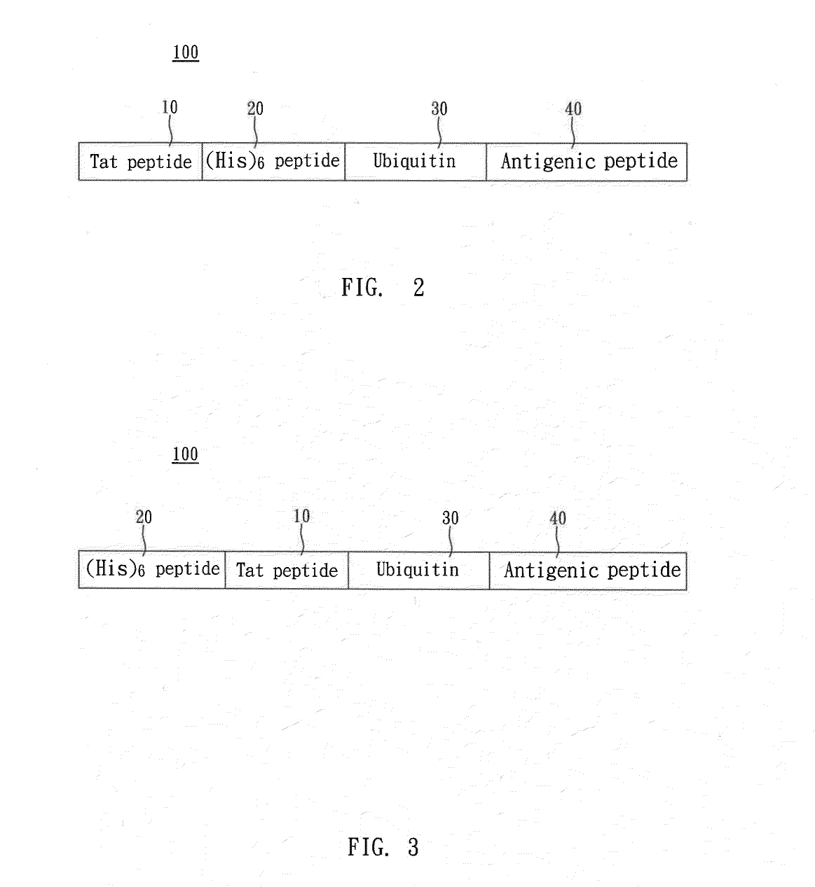

Regarding step (a), the sequence of the fusion protein 100 is provided in turn with a Tat-derived peptide 10, a (His)₆ peptide 20, a ubiquitin 30, and an antigenic peptide 40 from the N-terminal end to the C-terminal end, as shown in FIG. 2. Alternatively, the sequence is allowed to be provided in turn with a (His)₆ peptide 20, a Tat-derived peptide 10, a ubiquitin 30, and an antigenic peptide 40, as shown in FIG. 3. Therein, the sequence of the antigenic peptide 40 is allowed to be either RRFKEGGRGGKY or RRYLENGKETL. When the sequence thereof is RRFKEGGRGGKY, the antigenic peptide 40 is derived from the DNA primase of a Chlamydia trachomatis, and the fusion protein 100 thus formed is named as a THUC fusion protein 100. When the sequence thereof is RRYLENGKETL, the antigenic peptide 40 is derived from a human HLA-B27 heavy chain, and the fusion protein 100 thus formed is named as a THUB fusion protein 100.

The method of preparing the fusion protein 100 comprises: (I) preparing a cDNA of the fusion protein 100 and encoding the cDNA through PCR; (II) cloning the cDNA into a vector and transforming the vector into E. coli cells, whereby the fusion protein 100 is expressed in a large amount in the E. coli cells; (III) extracting the protein in the E. coli cells; and (IV) purifying the protein extracted in (III). Therein, based on the fact that the fusion protein 100 contains (His)₆ peptide 20, the fusion protein 100 is allowed to be easily purified by “Ni²⁺-Sepharose column”. Therefore, the purified fusion protein 100 extract is obtained by purifying the protein extracted by firstly the affinity chromatography using a Ni²⁺-Sepharose column and subsequently the cation exchange chromatography by a fast protein liquid chromatography (FPLC).

Regarding step (b), peripheral blood mononuclear cells (PBMCs) of patients with Ankylosing Spondylitis (AS) are extracted firstly. Afterward, adding the fusion protein 100 into the culture medium of the PBMCs for culturing the PBMCs (5 μm/ml of the fusion protein 100 added/per 5×10⁶ of cells), Based on known arts, the sequence of the Tat-derived peptide 10 is GRKKRRQRRR, which is section of peptide with dense positive electricity, whereby the Tat-derived peptide 10 is allowed to carry the fused protein across the cellular membrane to enter the cytosol (Adv Drug Deliv Rev. 57:559-577, 2005). Therefore, the fusion protein 100 enters from extracellular side of the PBMCs into the cytosol of the PBMCs by the help of the Tat-derived peptide 10.

Regarding step (c), with ubiquitin C-terminal hydrolases in the cytosol of the PBMCs which is capable of cleaving the C-terminal of the ubiquitin 30 accurately, the ubiquitin C-terminal hydrolase cleaves the antigenic peptide 40 of the present invention from the fusion protein 100.

Regarding step (d), the antigenic peptide 40 cleaved in (c) is transported into the endoplasmic reticulum by a TAP1/2 transporter on the membrane of the endoplasmic reticulum in the PBMCs.

Furthermore, the present invention also transports the fusion protein 100 into the C1R-B2704 cells, wherein the C1R-B2704 cells are a type of human lymphoid cell line, and the cellular surface thereof presents misfolded HLA-B27 heavy chain. After the fusion protein 100 of the present invention is delivered into the C1R-B2704 cells, the method and theory of the antigenic peptide 40 delivered into the endoplasmic reticulum are identical to the method and theory of the PBMCs delivery aforementioned.

The method and theory of the present invention are explained as above, and the effects of the present invention are explained hereinafter.

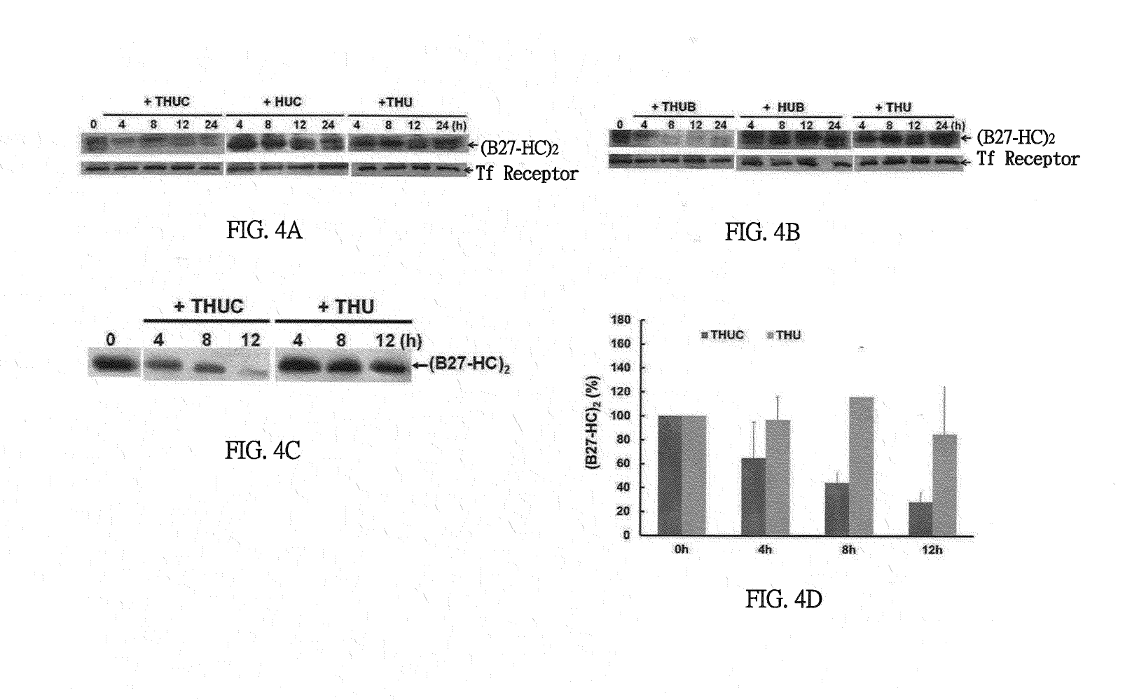

Referring to FIG. 4A to FIG. 4D, FIG. 4A and FIG. 4B illustrate the experimental statistics with the THUC fusion protein 100 and THUB fusion protein 100 delivered into C1R-B2704 cells, respectively; FIG. 4C and FIG. 4D illustrate the experimental statistics with the THUC fusion protein 100 and THUB fusion protein 100 delivered into PBMCs, respectively. The Transferrin receptor (Tf receptor) in both FIG. 4A and FIG. 4B serves as an internal control of the experiment, and the THU, HUB, and HUC are the comparison groups of the experiment. Therein, the THU is another fusion protein without antigenic peptide 40, and the HUB and HUC are other two different fusion proteins without a Tat-derived peptide 10. When the THUC fusion protein 100 of the present invention is delivered into the cells, the amount of the (B27-HC)₂ remarkably decreases, as shown in FIGS. 4A, 4C, and 4D. In addition, when the THUB fusion protein 100 is delivered into the cells, the same effect is achieved, as shown in FIG. 4B.

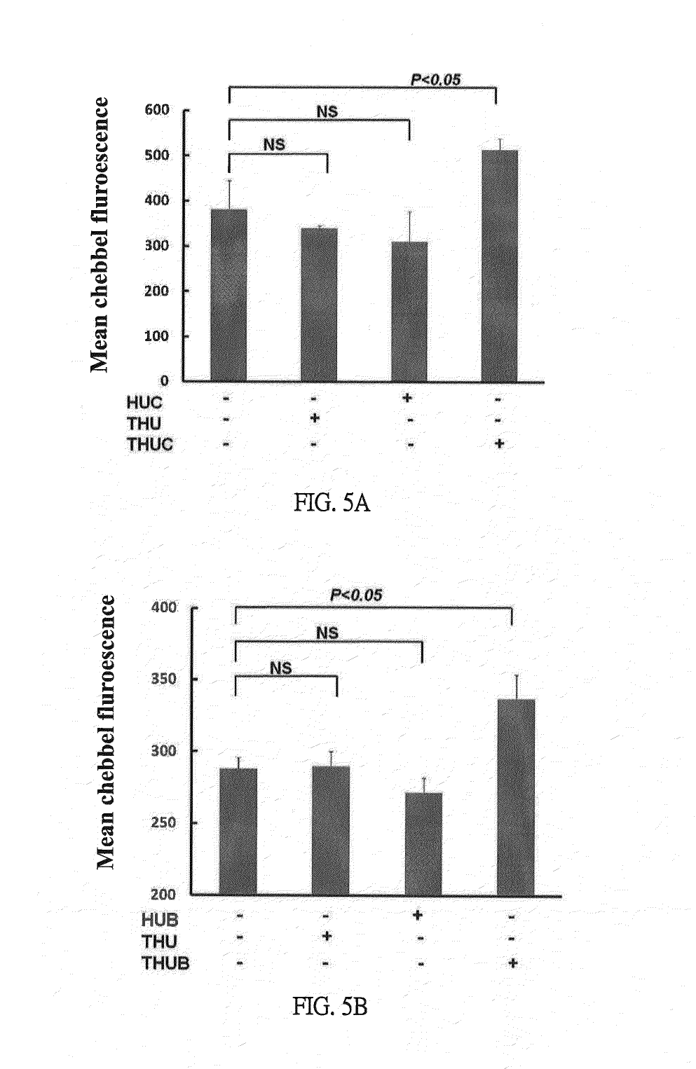

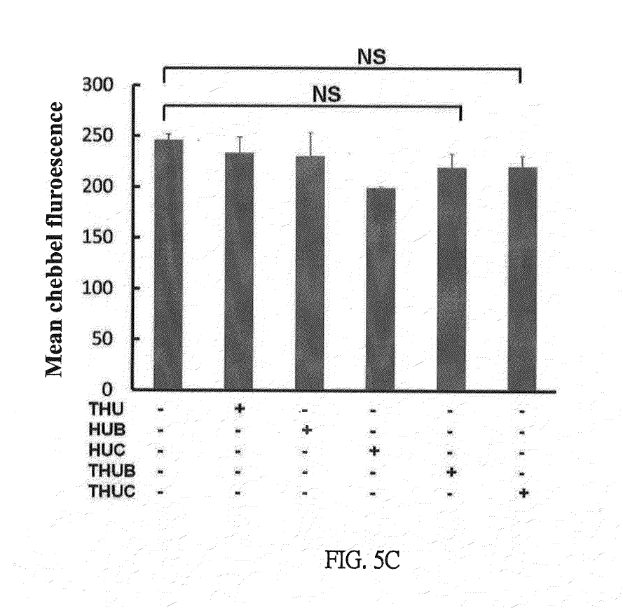

Referring to FIG. 5A to FIG. 5C, after the THUC fusion protein 100 and THUB fusion protein 100 of the present invention are delivered into the C1R-B2704 cells, either respectively or together, the amounts of the antigenic peptide 40 presented on the cell surface are determined by use of flow cytometry, respectively. Therein, the THU, HUB, and the HUC are comparison groups of the experiment. The statistics on the Y-axis represent the mean channel fluorescence (MCF) of W6/32 monoclonal antibody, wherein the W6/32 monoclonal antibody is able to be bound to the HLA-B27 heavy chain, which is complexed with the β₂ m on the surface of the C1R-B2704 cells. Therefore, the intensity of the mean channel fluorescence (MCF) of the W6/32 monoclonal antibody represents the amount of the correctly folded HLA-B27 heavy chains, and at the same time represents the amount of the antigenic peptide 40 complexed with HLA-B27 and presented on the surface of the C1R-B2704 cells.

After the THUC fusion protein 100 of the present invention is delivered into the C1R-B2704 cells, the amount of the antigenic peptide 40 presented on the cell surface of the C1R-B2704 cells increases remarkably, as shown in FIG. 5A. Also, after the THUB fusion protein 100 of the present invention is delivered into the C1R-B2704 cells, the same increasing effect is acquired, as shown in FIG. 5B. However, if the TAP1 shRNA is used to knockdown the TAP1 transporters on the endoplasmic reticulum of the C1R-B2704 cells, no matter which one of the THUC fusion protein 100 and THUB fusion protein 100 is delivered into the C1R-B2704 cells, the amount of the antigenic peptide 40 presented on the cell surface of the C1R-B2704 cells remains the same without increment, as shown in FIG. 5C.

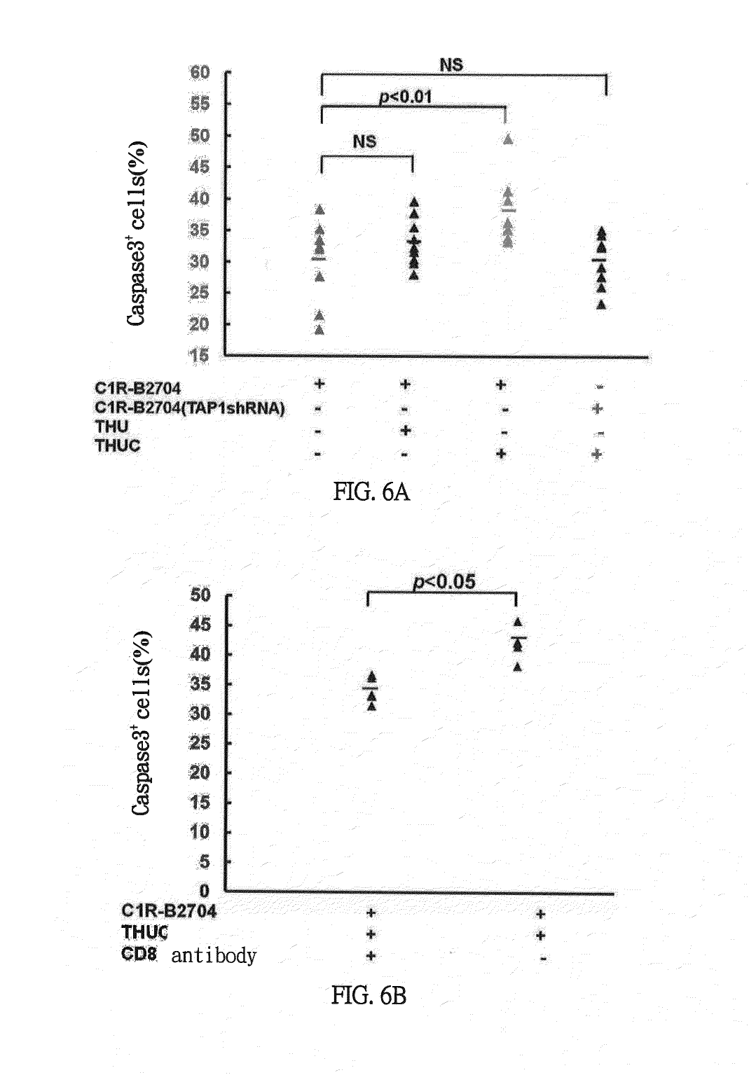

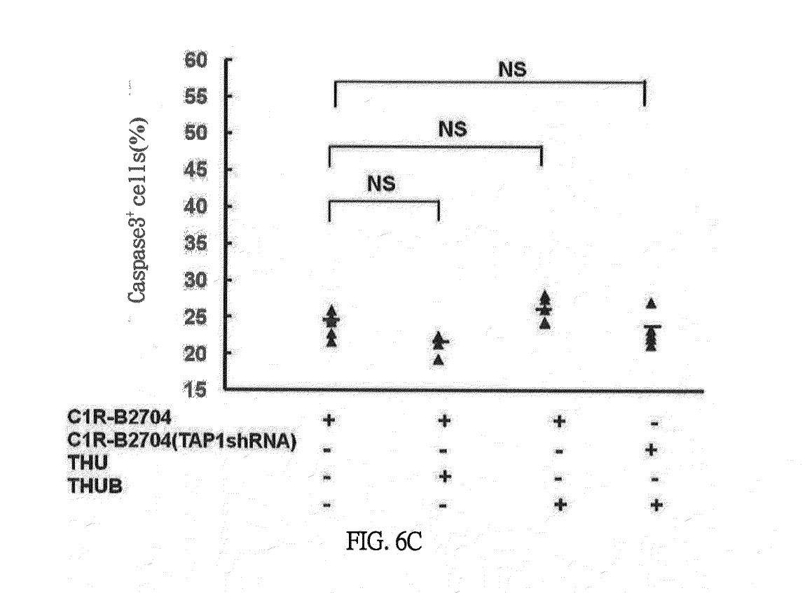

FIG. 6A to FIG. 6C are statistics diagrams illustrating the amounts of cells with apoptosis. The THUC fusion protein 100 and THUB fusion protein 100 are delivered into the C1R-B2704 cells, respectively, whereby the antigenic peptide 40 is presented on the cellular membrane of the C1R-B2704 cells, and then the CD8⁺ T cells activated by the antigenic peptide 40 is added. The amounts of the C1R-B2704 cells with apoptosis are determined by use of the flow cytometry. Therein, the activated CD8⁺ T cells are prepared by stimulating the PBMCs of patients of Ankylosing Spondylitis with interlukin-2 (IL-2) and the THUC fusion protein 100 or the THUB fusion protein 100, respectively, and afterward isolated from the PBMCs stimulated. THU is the comparison group. The TAP1 shRNA is used to knockdown the TAP1 transporters in the C1R-B2704 cells. In addition, the C1R-B2704 cells with apoptosis are marked with anti-active caspase 3 antibodies, and the relative statistics are presented on the Y-axis of FIG. 6A to FIG. 6C.

After the THUC fusion protein 100 of the present invention is delivered into the C1R-B2704 cells, amount of the C1R-B2704 cells with apoptosis increases remarkably, as shown in FIG. 6A, Such cellular apoptosis response is proved to be related to the activated CD8⁺ T cells, as shown in FIG. 6B. The activated CD8⁺ T cells trigger the cytotoxicity response, thereby causing the apoptosis of the C1R-B2704 cells. In contrast, when the THUB fusion protein 100 is delivered into the C1R-B2704 cells, amount of C1R-B2704 cells with apoptosis remains the same without increment, as shown in FIG. 6C.

According to the statistics shown in FIG. 6A to FIG. 6C, if the antigenic peptide 40 is derived from bacteria, the antigenic peptide 40 activates the CD8⁺ T cells and triggers cytotoxicity response. In contrast, if the antigenic peptide 40 is derived from the self-protein of the patient, the antigenic peptide 40 fails to activate the CD8⁺ T cells, thereby preventing the cytotoxicity response from happening.

To sum up, the present invention provides a method of delivering antigenic peptide 40 into the endoplasmic reticulum by the fusion protein, which transports the antigenic peptide 40 with accurate size and sequence into the endoplasmic reticulum for promoting the HLA-B27 heavy chain folding correctly in the endoplasmic reticulum. Afterward, the HLA-B27 heavy chain correctly folded is complexed with β₂-microglobin (β₂m) to form the HLA-B27, whereby the HLA-B27 is complexed with the antigenic peptide 40, and the antigenic peptide 40 is present on the cellular surface. As a result, the present invention decreases the amount of HLA-B27 heavy chain homodimer, (B27-HC)₂, thereby mitigating the inflammatory response. Therefore, the present invention is allowed to be applied to clinical researched of Ankylosing Spondylitis treatment.

Furthermore, if the bacteria-derived antigenic peptide 40 is delivered into human cells, the antigenic peptide 40 is presented on the cellular surface by the HLA-B27 of human cells, thereby activating the CD8⁺ T cells and further triggering the cytotoxicity response. Therefore, the present invention is allowed to be applied to the researches of cancer treatment. Therein, if a specific antigenic peptide is delivered into cancer cells by use of the method of the present invention, the MHC class I molecules of the cancer cells will present the antigenic peptide on the cellular membrane thereof. As a result, the cancer cells will be killed by the cytotoxicity response which is triggered by the activated CD8⁺ T cells, whereby the cancer is being treated.

What is claimed is:

1. A method of delivering antigenic peptide into the endoplasmic reticulum (ER) by a fusion protein, comprising following steps:
(a) preparing a fusion protein, wherein the fusion protein is provided with a Tat-derived peptide, a (His)₆ peptide, a ubiquitin, and an antigenic peptide;
(b) delivering the fusion protein across the cellular membrane into the cytosol by the Tat-derived peptide;
(c) cleaving the antigenic peptide from the fusion protein by cytosolic ubiquitin C-terminal hydrolases; and
(d) transporting the cleaved antigenic peptide into the endoplasmic reticulum by a TAP1/2 transporter on the endoplasmic reticulum membrane.

2. The method of claim 1, wherein the sequence of the fusion protein is provided in turn with the Tat-derived peptide, the (His)₆ peptide, the ubiquitin, and the antigenic peptide from the N-terminal end to the C-terminal end.

3. The method of claim 1, wherein the sequence of the fusion protein is provided in turn with the (His)₆ peptide, the Tat-derived peptide, the ubiquitin, and the antigenic peptide from the N-terminal end to the C-terminal end.

4. The method of claim 1, wherein the sequence of the antigenic peptide is RRFKEGGRGGKY.

5. The method of claim 1, wherein the sequence of the antigenic peptide is RRYLENGKETL.

6. A fusion protein provided with a Tat-derived peptide, a (His)₆ peptide, a ubiquitin, and an antigenic peptide, wherein the antigenic peptide is on the C-terminal end of the fusion protein and at the same time bound to the C-terminal end of the ubiquitin.

7. The fusion protein of claim 6, wherein the sequence of the fusion protein is provided in turn with the Tat-derived peptide, the (His)₆ peptide, the ubiquitin, and the antigenic peptide from the N-terminal end to the C-terminal end.

8. The fusion protein of claim 6, wherein the sequence of the antigenic peptide is RRFKEGGRGGKY.

9. The fusion protein of claim 6, wherein the sequence of the antigenic peptide is RRYLENGKETL.

10. A method of manufacturing a fusion protein, comprising following steps:
I. preparing a cDNA of the fusion protein and encoding the cDNA through PCR;
II. cloning the cDNA into a vector and transforming the vector into E. coli cells, whereby the fusion protein is expressed in a large amount in the E. coli cells;
III. extracting the protein in the E. coli cells; and
IV. purifying the protein extracted in step III to acquire the fusion protein.

11. The method of manufacturing a fusion protein of claim 10, wherein the protein is purified by firstly the affinity chromatography using a Ni2⁺-Sepharose column and subsequently the cation exchange chromatography by a fast protein liquid chromatography (FPLC).

12. The method of manufacturing a fusion protein of claim 10, wherein the fusion protein is provided with a Tat-derived peptide, a (His)₆ peptide, a ubiquitin, and an antigenic peptide, while the antigenic peptide is on the C-terminal end of the fusion protein and at the same time bound to the C-terminal end of the ubiquitin.

13. The method of manufacturing a fusion protein of claim 12, wherein the sequence of the fusion protein is provided in turn with the Tat-derived peptide, the (His)₆ peptide, the ubiquitin, and the antigenic peptide from the N-terminal end to the C-terminal end.

14. The method of manufacturing a fusion protein of claim 12, wherein the sequence of the antigenic peptide is RRFKEGGRGGKY.

15. The method of manufacturing a fusion protein of claim 12, wherein the sequence of the antigenic peptide is RRYLENGKETL.
